(19) United States
(12) Patent Application Publication
(10) Pub. No.: US 2024/0189379 A1
(43) Pub. Date: Jun. 13, 2024

(54) EDIBLE CANNABIS PLANT POWDER

(71) Applicant: The Hemp Tea Company, LLC, Montpelier, VA (US)

(72) Inventors: Chad Currin, Glenville, NY (US); Thomas E. Gilardi, Montpelier, VA (US)

(73) Assignee: The Hemp Tea Company, LLC, Montpelier, VA (US)

(21) Appl. No.: 18/534,272

(22) Filed: Dec. 8, 2023

Related U.S. Application Data
(60) Provisional application No. 63/431,232, filed on Dec. 8, 2022.

(51) Int. Cl. A61K 36/185; A23D 9/007; A23D 9/05; A61K 9/00; A61K 31/00; A61K 35/741; A61K 36/28; A61K 47/14

(52) U.S. Cl. CPC A61K 36/185; A23D 9/007; A23D 9/05; A61K 9/0056; A61K 31/658; A61K 35/741; A61K 36/28; A61K 47/14

(57) ABSTRACT

A composition of matter comprising an edible organically based substance, as well as an apparatus and method of making the composition of matter is disclosed. In one embodiment, the composition of matter, apparatus and method are directed to an edible organically based powder comprising an organic material, MCT oil, and MCT oil powder. In certain embodiments, the organic material and MCT oil are mixed together, heated and agitated binding the MCT oil to the organic material and decarboxylating the compound. The decarboxylated compound is then cooled. The decarboxylated compound is then ground to a desired size (which in some embodiments may be as fine as a powder) which is then mixed with MCT oil powder to produce the edible organic substance. In certain embodiments, the organic material comprises cannabis, and the resulting edible organic substance resulting from the disclosed process is an edible cannabis plant powder.

### CROSS REFERENCE TO RELATED APPLICATION

This application claims priority to and the benefit of U.S. Provisional Patent Application Ser. No. 63/431,232, entitled “EDIBLE CANNABIS PLANT POWDER” filed Dec. 8, 2022, the entirety of which is incorporated herein by reference.

### FIELD OF THE DISCLOSURE

The present embodiments are generally directed to organic materials for human ingestion. Such embodiments may include but not be limited to edible organics which are processed in such a manner that the person consuming such organics receive some or all of the beneficial components of the organic when the organic is ingested. The embodiments further disclose methods and apparatus for processing such organics and the manner for using such processed organics to achieve the intended health, nutritional and other benefits of ingesting the organic.

### BACKGROUND

Man has long sought to reap, and maximize, the health and nutritional benefits from various organic materials found in nature. One such organic which has been closely studied for its wide-ranging health and nutritional benefits is the cannabis plant.

One of the oldest means used to derive at least some of the benefits from the cannabis plant involves inhaling the smoke emanating from burning the cannabis plant material such as, for example, by smoking the cannabis plant material. However, such means of consumption also introduces certain health risks associated with smoking.

Another means to derive certain benefits from the cannabis plant involves ingesting edible forms of the cannabis plant material or components of the cannabis plant material. However, existing edibles typically include highly processed cannabinoid oils which are lacking cannabinoid diversity, terpenes, and other beneficial components of the cannabis plant.

Typically, humanly consumable edibles derived from certain organic materials are made by chemical or solvent extraction. In the case of the cannabis plant as the organic, certain cannabinoids including, but not limited to Tetrahydrocannabinol (“THC”), Cannabidiol (“CBD”), and/or other major and/or minor cannabinoids, are extracted from the cannabis plant material by chemical or solvent extraction. The extracted cannabinoids are then highly processed to isolate the extracted materials from the cannabis plant itself and converted into a form that may be processed by human digestion. However, though various cannabinoid extraction processes currently exist, no such process allows for the separation or isolation of cannabinoids from the cannabis plant without the use of chemical or solvent extraction methods resulting in edible materials which may provide a wide spectrum of health, nutritional and other benefits realized through the human consumption of such edible materials.

### SUMMARY OF THE INVENTION

The present invention relates to processes, methods and apparatus for producing edible compositions from organic materials and the compositions derived therefrom. In one embodiment, the invention relates to a process, method, and apparatus for producing an edible powder from a cannabis plant and compositions derived therefrom.

In one embodiment, an edible organically based powder comprising an organic material, a fat, such as for example, MCT (medium-chain triglyceride) oil which may comprise medium-chain triglycerides, and MCT oil powder. In certain embodiments, the organic material and MCT oil are mixed together, heated and agitated while heating, and cooled. The organic material and MCT oil mixture are then ground to a desired size (which in some embodiments may be as fine as a powder) which is then mixed with the MCT oil powder (also referred to herein as “MCT powder”) to produce the edible organic substance.

In another embodiment, the organically based material comprises cannabis and the resulting edible organic substance is an edible cannabis plant powder.

In yet another embodiment, a process for making an edible organic powder is disclosed. In one embodiment of the process comprises the steps of mixing together the organic material and MCT oil, heating the organic material and MCT oil mixture in a convection oven which is capable of decarboxylating the mixture while heating, agitating the organic material and MCT oil mixture while heating, cooling the organic material and MCT oil mixture after heating, grinding the cooled organic material and MCT oil mixture into a desired size (which in some embodiments may be as fine as a powder), and mixing the ground organic material and MCT oil mixture with an MCT oil powder to produce the edible organic substance.

In another embodiment of the process disclosed herein, the organically based material used in the process comprises cannabis and the resulting edible organic substance resulting from the process is an edible cannabis plant powder.

### BRIEF DESCRIPTION OF THE DRAWINGS

The accompanying drawings, diagrams, figures and/or Appendices, which are included to provide a further understanding of the invention and are incorporated in and constitute a part of this specification, illustrate embodiments of the invention and together with the description serve to explain the principles of the invention, in which like numerals refer to like components or steps, and wherein:

FIG. 1 is a flow diagram illustrating an embodiment of the process for making an edible organic powder of the present invention wherein the raw organic material comprises a cannabis plant material which is bound with a fat during decarboxylation.

APPENDIX A comprises pages from a testing laboratory report which confirms the composition of a sample of one embodiment of a finished cannabis powder produced by process disclosed herein.

### DETAILED DESCRIPTION OF THE INVENTION

It is to be understood that the figures and descriptions of the present invention have been simplified to illustrate elements that are relevant for a clear understanding of the present invention, while eliminating, for the purposes of clarity, other elements or steps typically found in the prior art. Those of ordinary skill in the pertinent art may recognize that other elements and steps may be desirable and/or required in order to implement the present invention. However, because such elements and steps are well known in the art, and because they do not facilitate a better understanding of the present invention, a discussion of such elements and steps may not be provided herein.

Reference will now be made in detail to several exemplary and non-limiting embodiments of the present invention, some of which are illustrated in the accompanying drawings.

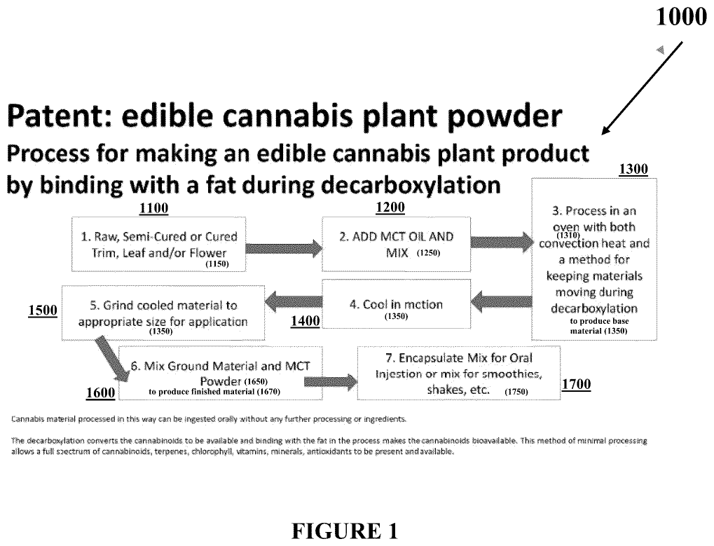

Turning now to FIG. 1, there is shown a process flow diagram depicting an embodiment of the method and/or process 1000 of the present invention. In particular, this embodiment of process 1000 depicts one method for making an edible cannabis plant product by binding an organic material with a fat during decarboxylation.

Process 1000 begins with preparation 1100 of raw organic material 1150 such as for example shucked cannabis, cannabis flower and/or cannabis trim. Depending upon the intended end use, raw organic material 1150 may comprise regionally available medicinal plants.

At 1200, between about ½% to about 1% by mass of fat 1250, such as liquid MCT oil, which MCT oil may typically comprise medium-chain triglycerides, is added to raw material 1150.

At 1300, the combined raw material 1150 and fat 1250 are placed and mixed in dehydrator 1310. Dehydrator 1310 is capable of heating and keeping raw material 1150 and fat 1250 in motion during process 1000, which generally causes the decarboxylation of raw material 1150 and fat 1250. In certain embodiments, dehydrator 1310 functions with one or more characteristics of a sealed convection oven and/or a food waste dehydrator. In one embodiment, dehydrator 1310 may comprise a dehydrator manufactured by ENIC, Model Ecovim Eco-66, which uses a combination of convection heating and air flow which results in the decarboxylation of raw material 1150 and fat 1250.

In other embodiments, dehydrator 1310 may comprise food waste dehydration machines which employ grinding and/or heat to pulverize and/or dehydrate combined raw material 1150 and fat 1250 such as FoodCycler Eco 3 Model Numbers FC-200 and FC-200E, FoodCycler Eco 5 Model Numbers FC-100 and FC-100E, and FoodCycler FC-50 each manufactured and/or distributed by Food Cycle Science Corporation of Ottawa, Ontario Canada (see e.g. https://foodcycler.com incorporated herein by reference), and Model Numbers TG-CC-5, TG-CC-10, TG-CC-50, TG-CC-100, TG-CC-200, TG-CC-300, TG-CC-500, TG-CC-1000, TG-CC-2000, TG-CC-5000, and TG-CC-10T each manufactured and/or distributed by WuXi TOGO Environment Equipment Co., Ltd. of Southern District of Wanshi Industrial Park, Yixing, Jiangsu, China (see e.g. https://togosolution.com incorporated herein by reference).

In yet other embodiments, dehydrator 1310 may comprise food waste dehydration machines which employ grinding and/or heat to pulverize and/or dehydrate combined raw material 1150 and fat 1250 available from Hungry Giant Waste Systems of Austin, Texas (see https://hungrygiantrecycling.com incorporated herein by reference), Somat Waste Reduction Solutions of Lancaster, Pennsylvania (see https://somatcompany.com incorporated herein by reference), and Ecovrs Recycling Solutions of Athens, Greece (see https://www.ecovrs.com incorporated herein by reference).

During step 1300, raw materials 1150 and fat 1250 are processed by heating and mixing in dehydrator 1310 for approximately one hour while gradually increasing the heat in convection oven 1310 to between about 83° C. and 90° C. In certain embodiments, maintaining the heat in dehydrator 1310 containing raw materials 1150 and fat 1250 for approximately fifteen (15) minutes at the highest level of the heat range may be preferred. Optimal heating duration for any given raw material 1150 and fat 1250 mixture, in part, may depend upon the actual moisture content of raw material 1150. Typically, however, many raw materials 1150 and fat 1250 mixtures require approximately a minimum of one (1) hour of heating and mixing in dehydrator 1310 and/or at least until a desired moisture level is achieved. An acceptable moisture level in raw material 1150 and fat 1250 mixture may typically be about 5% by mass, however moisture levels in certain embodiments of between about 3% and about 8% by mass may also be acceptable.

Typically, steps 1200 and 1300 bind fat 1250 (such as, for example, MCT oil 1250) to raw material 1150 and decarboxylates raw material 1150. In the case where raw material 1150 is cannabis, decarboxylation generally converts the major and minor phytocannabinoids (e,g, THC-A and CBD-A, respectively) in cannabis 1150 into active molecular forms (e.g. THC-A in cannabis 1150 and THC in the base material 1350 ultimately produced from process 1000) which may influence a human's neurotransmitters.

In certain embodiments, the decarboxylation process converts the cannabinoids present in raw organic material 1150 such that the cannabinoids are made available to bind with fat 1250 during process 1000, and/or making the cannabinoids bioavailable in products produced by process 1000. In addition, in certain embodiments, process 1000 may allow for a full spectrum of certain cannabinoids, terpenes, chlorophyll, vitamins, antioxidants, to be present and available in products produced by process 1000.

Once the mixing 1200 and processing 1300 steps are completed, raw material 1150 and fat 1250 mixture may be cooled by cooling process 1400, which results in the production of base material 1350. In certain embodiments, cooling process 1400 may comprise continuous tumbling, mixing or other motion, and/or may cool raw material 1150 and fat 1250 mixture to a temperature of about 20° C. or about room temperature. Such substantially continuous motion during process 1000 may assist in minimizing, and preferably preventing, raw material 1150 and fat 1250 mixture and/or base material 1350 from coming in contact with or otherwise touching the heat source introduced at step 1300, and/or from burning.

Once base material 1350 is sufficiently cooled at 1400, base material 1350 may be ground at 1500 into an appropriate size 1550, which may be any size appropriate for the intended use of the product produced by process 1000. In one embodiment, base material 1350 may be ground 1500 into a powder 1550 such as, for example, a powder 1550 to suit the product to be produced by process 1000. In certain embodiments, base material 1350 may be ground 1500 into an appropriate particle size 1550 which promotes the rapid and/or efficient ingestion, absorption and/or other processing by the human body. Additionally, or alternatively, in certain embodiments, base material 1350 may be ground 1500 to an appropriate particle size 1550 which maximizes the beneficial characteristics of the product produced by process 1000 to be realized from the ingestion, absorption and/or other processing by the human body of the product produced by process 1000. For example, such beneficial characteristics may include, but not be limited to, health, nutritional and other benefits.

Once ground material 1550 has been ground to the desired size at 1500, ground material 1550 may be mixed at 1600 with at least approximately 10% by mass of powdered MCT oil 1650. In certain embodiments, ground material 1550 may be mixed at 1600 with between about 15% and about 50% by mass of powdered MCT oil to produce finished material 1670.

In certain embodiments, varying the amount of powdered MCT oil 1650 added to ground material 1550 may increase or decrease the digestibility of finished material 1670. In addition, or alternatively, in certain embodiments, varying the amount of powdered MCT oil 1650 added to ground material 1550 may increase or decrease the uptake “ability and speed” realized from the ingestion, absorption and/or other processing by the human body of finished material 1670. In certain embodiments, adding above about 15% by mass of powdered MCT oil 1650 added to ground material 1550 may be used to maximize THC levels in finished material 1670.

After mixing ground material 1550 with appropriate amount of powdered MCT oil 1650 at 1600 to produce finished material 1670, finished material 1670 may be used for ingestion, absorption and/or other processing by the human body, including but not limited to, as an edible product. In certain embodiments, finished material 1670 may be in the form of an edible powder 1670.

The characteristics of finished material 1670 may be varied as different types and varying amounts of raw organic materials 1150, fats 1250, and/or fat powders 1650 are used in process 1000. The characteristics of finished material 1670 also may be varied as different heating and cooling temperatures and durations, agitation speeds and durations are employed in process 1000.

In one embodiment, where cannabis is used as raw organic material 1150 in process 1000, resulting finished material 1670 may have a higher concentration of THC and/or lesser or no amounts of CBD.

In another embodiment, where hemp is used as raw organic material 1150 in process 1000, resulting finished material 1670 may have a higher concentration of CBD and/or lesser or no amounts of THC.

At, 1700, edible powder 1670 may be packaged and ingested as a ready-to-eat product itself, sprinkled on food, and/or mixed into a beverage such as a smoothie. In addition, at 1700, edible powder 1670 may be placed in a capsule 1750, pressed into a tablet 1750, and/or otherwise used in any ingestible fashion or form.

Finished material 1670 may also be mixed with other organics, compositions, and/or products such as CBD, echinacea, probiotics and the like, in order to meet the intended end characteristics of finished material 1670.

In certain embodiments, raw organic material 1150, and particularly cannabis material, processed in accordance with the disclosure herein, may be orally ingested by humans without further processing or ingredients.

In yet other embodiments, finished material 1670 may be adapted for use as an alternative medicinal to commercially available products which assist individuals in the withdrawal from or reduction of use of traditional pain management treatments such as opioids.

Appendix A hereto comprises a testing laboratory report which confirms the composition of a sample of one embodiment of finished cannabis powder 1650 produced by process 1000 disclosed herein.

In this embodiment, approximately six (6) lbs (approximately 2750 grams) of cannabis trim and flower 1150 were prepared at 1100 and mixed at 1200 with approximately thirty (30) grams of MCT oil 1250 to form a cannabis 1150 and MCT oil 1250 mixture. In this embodiment, MCT oil 1250 comprised VERANDA brand coconut MCT Oil which comprises 100% coconut derived kosher, food grade, vegan, non-GMO fractionated coconut oil.

The combined cannabis material 1150 and MCT oil 1250 was then placed in dehydrator 1310 and heated and mixed for approximately one (1) hour and five (5) minutes by gradually increasing the heat to a maximum of about 90° C.

The cannabis material 1150 and MCT oil 1250 mixture was then cooled to approximately 20° C. by tumbling the cannabis 1150 and MCT oil 1250 mixture in dehydrator 1310 to produce base material 1350.

Base material 1350 was then ground into ground cannabis powder 1550.

Ground cannabis powder 1550 was then mixed with approximately 15% certified organic, vegan, keto certified, paleo certified, non-GMO, MCT oil powder obtained from GARDEN OF LIFE® (www.gardenoflife.com) to form finished cannabis powder 1650.

A sample of finished cannabis powder 1650 was then analyzed to determine the composition of finished cannabis powder 1650. The results of the composition analysis of the sample of finished cannabis powder 1650 are set forth in Appendix A hereto.

The disclosure herein is directed to the variations and modifications of the elements and methods of the invention disclosed that will be apparent to those skilled in the art in light of the disclosure herein. Thus, it is intended that the present invention covers not only the embodiments which are disclosed herein but also all modifications and variations of those embodiments and all equivalents thereof.

What is claimed is:

1. An edible organically based substance comprising:
an organic material;
MCT oil; and
MCT oil powder.

2. The edible organically based powder of claim 1, wherein said edible organic substance is an edible cannabis plant powder.

3. The edible organically based powder of claim 1, wherein said organic material comprises cannabis.

4. The edible organically based powder of claim 1, wherein said organic material comprises one or more of shucked cannabis, cannabis flower and cannabis trim.

5. The edible organically based powder of claim 1, wherein said organic material comprises hemp.

6. The edible organically based powder of claim 1, wherein said edible organically based powder comprises between about ½% and about 1% by mass of MCT oil.

7. The edible organically based powder of claim 1, wherein said MCT oil comprises medium-chain triglycerides.

8. The edible organically based powder of claim 1, wherein said edible organically based powder comprises at least about 10% by mass MCT oil powder.

9. The edible organically based powder of claim 1, wherein said edible organically based powder comprises between about 15% and about 50% by mass of MCT oil powder.

10. The edible organically based powder of claim 1, wherein said edible organically based powder comprises above about 15% by mass of MCT oil powder.

11. The edible organically based powder of claim 1, wherein said edible organically based powder comprises a higher concentration of THC and a lesser concentration of CBD.

12. The edible organically based powder of claim 1, wherein said edible organically based powder comprises THC and substantially no CBD.

13. The edible organically based powder of claim 1, wherein said edible organically based powder comprises a higher concentration of CBD and a lesser concentration of THC.

14. The edible organically based powder of claim 1, wherein said edible organically based powder comprises CBD and substantially no THC.

15. The edible organically based powder of claim 1, further comprising one or more of CBD, echinacea, and probiotics.

16. The edible organically based powder of claim 1 contained in a capsule.

17. The edible organically based powder of claim 1 substantially in the form of a tablet.

18. A process for making an edible organic substance comprising the steps of:
mixing together an organic material and an MCT oil to form an organic material and MCT oil mixture;
heating and agitating said organic material and MCT oil mixture in a dehydrator which is capable of decarboxylating said organic material and MCT oil mixture while heating and agitating;
after said heating and agitating, cooling said organic material and MCT oil mixture to produce a base material;
after said cooling, grinding said base material into a desired size to produce a ground base material; and
after said grinding, mixing said ground base material with an MCT oil powder to produce said edible organic substance.

19. The process for making the edible organic substance of claim 18, wherein said organically based material comprises cannabis, and said edible organic is an edible cannabis plant powder.

20. An edible organically based substance comprising:
an organic material;
MCT oil; and
MCT oil powder.
wherein said organic material and MCT oil are mixed together, heated and agitated while heating to produce an organic material and MCT oil mixture, wherein said organic material and MCT oil mixture are cooled to produce a base material, wherein said base material is then ground to a desired size to produce a ground base material, and wherein said ground base material is then mixed with an MCT oil powder to produce said edible organic substance.
